(19) United States
(12) Patent Application Publication
(10) Pub. No.: US 2025/0335004 A1
(43) Pub. Date: Oct. 30, 2025

(54) MOUNTABLE VIRTUAL REALITY STATION AND SYSTEM

(71) Applicant: David Black, Annapolis, MD (US)

(72) Inventor: David Black, Annapolis, MD (US)

(21) Appl. No.: 19/191,665

(22) Filed: Apr. 28, 2025

Related U.S. Application Data
(60) Provisional application No. 63/639,169, filed on Apr. 26, 2024.

(51) Int. Cl. G06F 1/16; A61L 2/10

(52) U.S. Cl. CPC G06F 1/163; A61L 2/10; A61L 2202/11; A61L 2202/122

(57) ABSTRACT

A virtual reality station includes a booth having at least one side wall and a mountable virtual reality system. The booth is configured to have an entrance and an inner chamber that can house at least one person. The mountable virtual reality system is arranged on at least one side wall of the booth and includes a unit body having a plurality of walls. The unit body has a front side and a back side, with a touch screen and a headset mounting display arranged on the front side and a power source which connects to the unit body. Preferably, the unit includes a system indicator display, wherein the system indicators are at least one of a power indicator, a Wi-Fi indicator, and a Bluetooth indicator.

### BACKGROUND OF THE DISCLOSURE

The present disclosure relates generally to virtual reality (VR) devices, and specifically to a booth with a mountable virtual reality station and system.

Virtual reality equipment typically involves a headset including video screens and speakers, among other devices. Virtual reality units can further include motion sensing devices to add to the experience and more accurately portray real-world situations.

When not in use, virtual reality equipment is often stored in a portable container, such as a bag or pouch. This is beneficial for an individual who owns the equipment, but it is not ideal for multi-user equipment, such as virtual reality equipment used by third parties in public settings. Virtual reality equipment used in a public setting is often done in an open space and often requires the user to stand. This arrangement lacks privacy and convenience, and can even be a safety hazard without a human attendant to ensure that the user stays within the bounds of the space.

Virtual reality is evidenced to provide an effective training modality; however, research has also shown that the more effective presence is experienced in a Virtual Environment (VE), the greater likelihood that participants will behave in a manner similar to their circumstances in real life. As VR applications serve training functions for a range of professionals, innovations that can aid in enhancing presence add an important innovation to enhance the efficacy of VR delivered applications. (See “Audio in VR: Effects of a Soundscape and Movement-Triggered Step Sounds on Presence”; Frontiers in Robotics AI, February 2020, Volume 7, Article 20.)

Research has shown that users of Virtual Reality environments find discrepancies between inputs (“sensory discrepancy”) coming from Physical Environments (PA) and Virtual Environments, producing a stress response. The desired presence of a VR-based experience can be influenced by the ambient physical environment and consideration should be given to innovations that reduce sensory discrepancy. (See Lécuyer, A. Playing with Senses in VR: Alternate Perceptions Combining Vision and Touch. IEEE Comput. Graph. Appl. 2017, 37, 20-26; Nordahl, R.; Nilsson, N.C. The Sound of Being There: Presence and Interactive Audio in Immersive Virtual Reality. In The Oxford, Handbook of Interactive Audio; Oxford Handbooks; Oxford University Press: Oxford, UK, 2014; Larsson, P.; Väljamae, A.; Västfjäll, D.; Tajadura-Jiménez, A.; Kleiner, M. Auditory-Induced Presence in Mixed Reality Environments and Related Technology. In The Engineering of Mixed Reality Systems; Dubois, E., Gray, P., Nigay, L., Eds.; Human-Computer Interaction Series; Springer: London, UK, 2010; pp. 143-163.)

There is thus a need for public-use devices that provide virtual reality capabilities with increased VR experiences over those known, as well as a need to provide privacy and convenience to users.

### SUMMARY OF THE DISCLOSURE

Accordingly, it is an object of the present disclosure to provide a virtual reality station including a booth having at least one side wall and a mountable virtual reality system. The booth is configured to have an entrance and an inner chamber that can house at least one person. The mountable virtual reality system is arranged on at least one side wall of the booth and includes a unit body having a plurality of walls. The unit body has a front side and a back side, with a touch screen and a headset mounting display arranged on the front side and a power source which connects to the unit body. Preferably, the unit includes a system indicator display, wherein the system indicators are at least one of a power indicator, a Wi-Fi indicator, and a Bluetooth indicator.

In one embodiment, the unit further includes a virtual reality headset tether connected with the unit body and configured for connection with a virtual reality headset.

In a second embodiment, the unit further includes a pair of side walls with an access panel and/or a cooling device arranged on one of the side walls.

In another embodiment, the unit further includes a mounting assembly configured for connection with a railing system.

In yet another embodiment, the unit further includes an eye tracking device and motion sensors configured to track movement.

In another embodiment, the unit further includes digital memory, at least one computer processor, and machine-readable instructions configured to integrate the station and system with devices, for instance personal biometric devices.

In a further embodiment, the unit further includes a sterilization unit arranged on the front wall including a plurality of walls defining an inner chamber. The sterilization unit preferably includes a UVC light configured to clean and store a virtual reality headset and a locking mechanism to secure a headset.

### BRIEF DESCRIPTION OF THE FIGURES

Other objects and advantages of the disclosure will become apparent from a study of the following specification when viewed in the light of the accompanying drawing, in which:

FIG. 1 is a front perspective view of a mountable virtual reality system according to the disclosure;

FIG. 2 is a side view of the embodiment of FIG. 1;

FIG. 3 is a perspective view of the embodiment of FIG. 1;

FIG. 4 is a perspective view of a booth according to the disclosure;

FIG. 5 is a front view of a mountable virtual reality system arranged on a front wall of a booth;

FIG. 6 is a front view of a mountable virtual reality system arranged on a side wall of a booth;

FIG. 7 is a perspective view of a booth with a mountable virtual reality system arranged on a front wall of a booth; and

FIG. 8 is a perspective view of a booth with a mountable virtual reality system arranged on a top wall of the booth.

### DETAILED DESCRIPTION

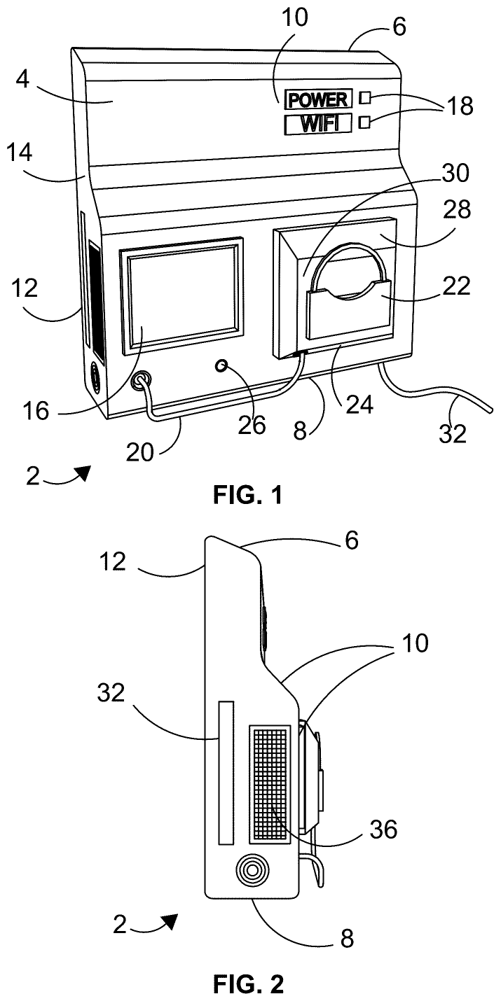

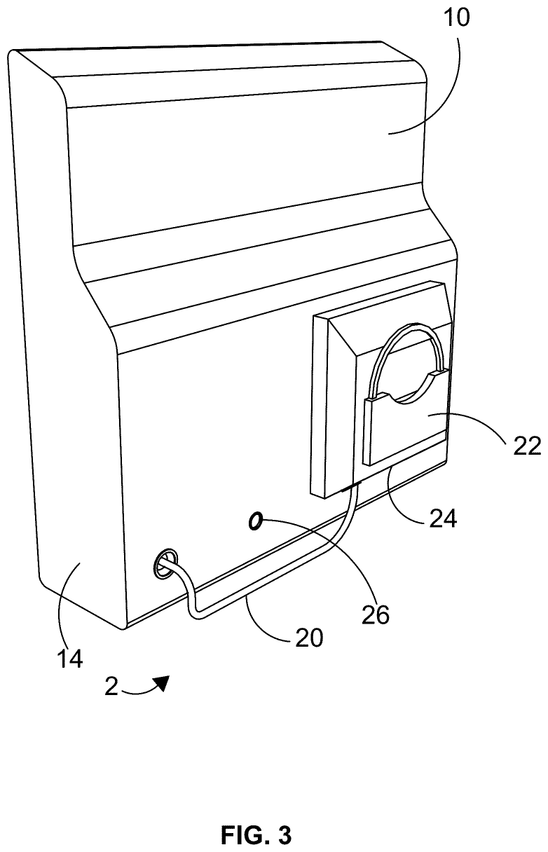

FIGS. 1-3 show a preferred embodiment of the mountable virtual reality system 2 of the present disclosure, which includes a hard casing unit 4 that houses hardware elements and includes top 6, bottom 8, front 10, rear 12 and side 14 walls. The housing unit is adapted to be mountable with a wall in a booth, preferably via a mounting assembly (not shown), by attaching the rear wall with a railing system that can be connected to a booth wall. Alternatively, the system could be integral to the booth.

The front wall 10 includes a touchscreen 16 with device navigation for users to select software applications, enter personal information, or otherwise customize their VR experience. The front wall further includes displays 18 that indicate power, Wi-Fi, and other connected device statuses (e.g. Bluetooth) to provide users with visibility into the security connection and operating status of the system.

A tether 20 connects a VR headset 22 to the front wall 10 of the main housing unit 2 to provide both a security measure and power source for the VR head mounted display (HMD) unit 24. The HMD unit includes at least one display, lenses and semi-transparent mirrors embedded in eyeglasses, and a visor or helmet. Preferably, the unit also includes eye tracking technology and motion sensors for tracking a user's movement, for instance hand movement.

The system further includes hardware, such as a CPU (not shown), arranged within the housing unit 2, which includes software programming to run the system and integrate the station equipment with wearable devices, preferably via a Bluetooth connection. There is also a camera and/or motion sensor 26 that provides hand tracking integration for user interface navigation and selection capabilities while wearing the VR headset 22.

A sterilization and housing unit 28 for the VR headset 22, which contains a chamber 30 to store and sanitize the VR headset between uses, is arranged on the front wall 10 of the housing unit 2. The chamber is preferably accessible via a lockable door (not shown). The headset is stored in this chamber and sanitized by a UVC light (not shown) arranged within the chamber of the sterilization and housing unit. Other methods for sanitizing the headset are contemplated, for instance by use of a sanitizing mist and/or sanitizing wipes.

The system is preferably powered via a 110v external power cord connection 32. However, wireless methods of power are also contemplated, such as a rechargeable battery.

To access the hardware, such as the CPU, touch screen, motion sensor, etc., for repairs and/or replacement, an access panel 34 including a removable cover is located on one of the side walls 14. The station and system also preferably includes a cooling device 36 such as a venting panel or fan to reduce the temperature of the unit.

To use the station and system, the mounting assembly is connected with a booth wall, preferably via a mounting rail, and the power cord 32 is connected with a power outlet. A user can then interact with the touchscreen 16 to choose a specific VR program, remove the VR headset 22 from the housing unit 28, wear the headset and use the VR software. While using the system, the user will have the option of connecting user devices with the system via Bluetooth, Wi-Fi or another wireless/wired connection. This will allow the user to synchronize user data and/or programs with the system as desired by the user or required by specific programs. When the user is finished, the VR headset 22 is once again placed in the housing unit 28 and sanitized for future use. The system can be powered down or remain on for another user. Alternatively, the station and system can be removed from the booth wall it is mounted on and moved to another location for mounting and use. For instance, in a large setting such as an office or school, multiple units of the VR system could be available and distributed to multiple users (e.g. employees, students) who can bring the unit with them into a booth, arrange the unit on a mount, and use the system while in the booth. When the user is finished, the user would remove the unit and leave the booth. The unit might be maintained by the user or returned to a designated storage and maintenance area.

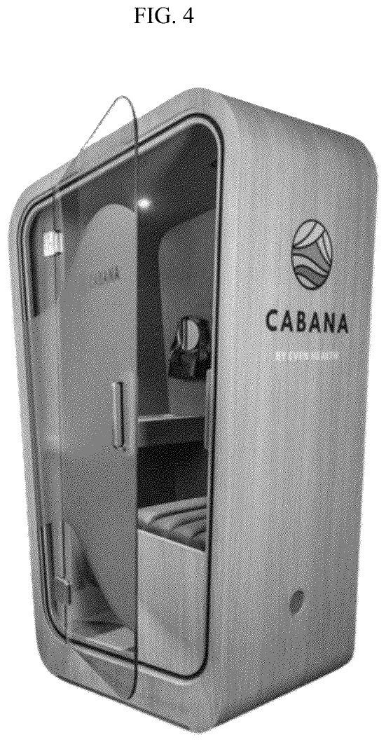

FIG. 4 shows a booth with a unit arranged on a side wall therein. The booth includes a seat, a ledge, a door, a rear wall, a front wall, and a side wall. In alternate embodiments not shown herein, the ledge or the seat may be foldable or slidable to allow the user to stand while inside. For the present embodiment, the ledge is fixed to the front wall. It will be understood by those with skill in the art that the able could be attached to the rear wall, the side wall, or the seat. The door includes an opaque glass to allow for sunlight but to maintain privacy for a user. In other embodiments, the door is replaced by a curtain, a sliding panel or other similar entrance. Moreover, the door may be constructed with metal, plastic, or a translucent glass. In the embodiment of FIG. 4, the rear wall, front wall, and side wall are curved with rounded corners, but other embodiments may have additional or fewer walls, which may form a variety of shapes such as domes, spheres, cubes, or polygons.

As is shown, the mountable virtual reality system 2 is arranged on the side wall of the booth. In this embodiment, the system is permanent. A user using the booth and device would enter the booth, close the door for privacy, power on the system and use the virtual reality headset as desired. When finished, the user would power down the system, replace the headset and exit the booth leaving the system for another user.

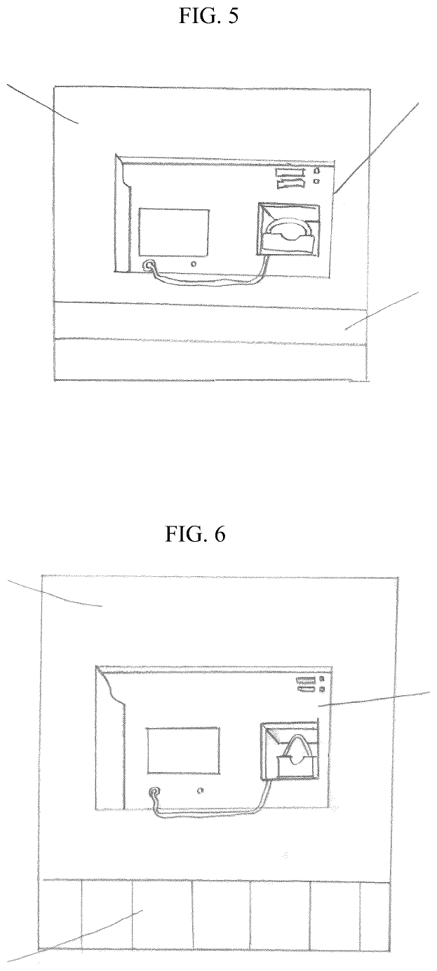

FIGS. 5 and 6 show embodiments of the mountable virtual reality system arranged on a front wall above the ledge and side wall above the seat, respectively. The mountable virtual reality system is preferably mounted within easy reach of the user from either a sitting or standing position, but other locations such as under or behind the seat are also contemplated. As noted above, the system could be fixed in place or could be removable, allowing for the system to mounted in multiple configurations depending on the intended use. For instance, in one embodiment, the booth could be constructed such that the system unit is mountable in any of the front, rear, side, or top walls or other locations in the booth.

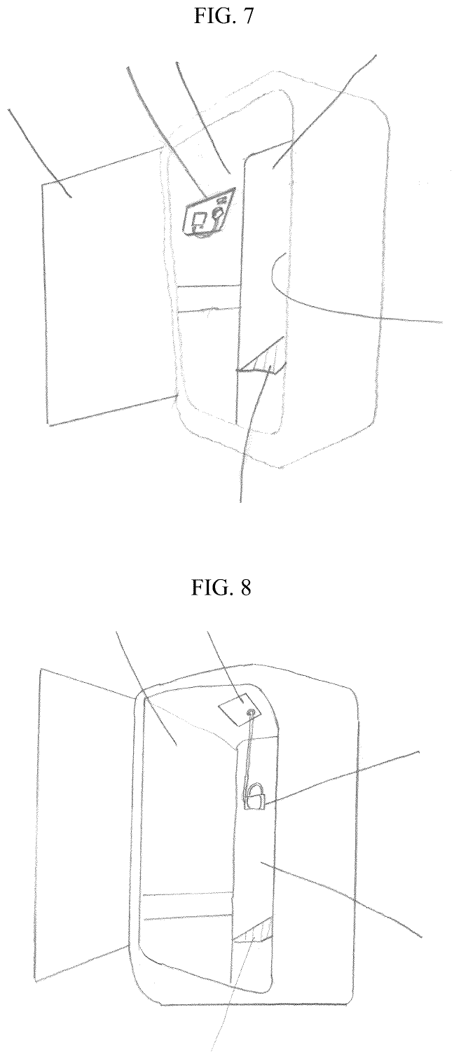

FIGS. 7 and 8 show two of the above-noted embodiments of the virtual reality station. In the embodiment of FIG. 7, the mountable virtual reality system is arranged on the front wall above the ledge and front of the intended position of a user. This embodiment situates the mountable virtual reality system within easy reach of the user and allows the VR headset to be placed on the ledge when not in use. FIG. 8 shows a preferred embodiment wherein the mountable virtual reality system is arranged on the top wall of the booth. This embodiment allows the main housing unit to be kept out of the way of the user, allowing them to move more freely. In this embodiment, the VR headset can hang down by the tether, which in some embodiments may be retractable. Other embodiments may integrate the mountable virtual reality system into the ledge, seat, or door.

Although the above description is in reference to a particular embodiment, it is to be understood that the embodiment is merely illustrative of the principles and applications of the present disclosure. It is therefore to be understood that numerous modifications may be made to the illustrative embodiment and that other arrangements may be devised and employed without departing from the spirit and scope of the present disclosure.

What is claimed is:

1. A virtual reality station comprising:
(a) A booth having at least one side wall defining an inner chamber and at least one entrance, the inner chamber configured to house at least one person;
(b) at least one virtual reality system configured for arrangement within the booth, the at least one virtual reality system comprising:
(i) a unit body having at least one wall;
(ii) a headset mount; and
(iii) a power source.

2. The virtual reality station defined in claim 1, wherein the at least one virtual reality system is configured for mounting on a booth wall.

3. The virtual reality station defined in claim 2, wherein the at least one virtual reality system is configured for mounting on the at least one side wall.

4. The virtual reality station defined in claim 1, wherein the at least one virtual reality system includes a plurality of mountable virtual reality systems.

5. The virtual reality station defined in claim 1, wherein the at least one virtual reality system further comprises a touch screen arranged on the unit body.

6. The virtual reality station defined in claim 1, wherein the at least one virtual reality system further comprises a virtual reality headset tether connected with the unit body configured for connection with a virtual reality headset.

7. The virtual reality station defined in claim 1, wherein the at least one virtual reality system further comprises a pair of side walls, one side wall including at least one of an access panel and a cooling device.

8. The virtual reality station defined in claim 1, wherein the at least one virtual reality system further comprises a mounting assembly configured for connection with a railing system.

9. The virtual reality station defined in claim 1, wherein the at least one virtual reality system further comprises a display arranged on the front wall, the display including system indicators, wherein the system indicators are at least one of a power indicator, a Wi-Fi indicator, and a Bluetooth indicator.

10. The virtual reality station defined in claim 1, wherein the at least one virtual reality system further comprises an eye tracking device and motion sensors configured to track movement.

11. The virtual reality station defined in claim 1, wherein the at least one virtual reality system further comprises digital memory, at least one computer processor, and machine-readable instructions configured to integrate the station and system with devices.

12. The virtual reality station defined in claim 1, wherein the at least one virtual reality system further comprises a sterilization unit arranged on the front wall including a plurality of walls defining an inner chamber, the sterilization unit being configured to clean and store a virtual reality headset.

13. The virtual reality station defined in claim 8, the sterilization unit having a locking mechanism.

14. The virtual reality station defined in claim 8, the sterilization unit including a UVC light.

15. The virtual reality station defined in claim 1, the power source including one of a power cord and battery.
